(19) United States
(12) Patent Application Publication
(10) Pub. No.: US 2009/0210038 A1
(43) Pub. Date: Aug. 20, 2009

(54) Medical Light Diffusers for High Power Applications and their Manufacture

(76) Inventors: Wolfgang Neuberger, F. T. Labuan (MY); Stefan Spaniol, Bonn (DE)

Correspondence Address: BOLESH J. SKUTNIK;CERAMOPTEC INDUSTRIES, INC., 515 SHAKER RD., EAST LONGMEADOW, MA, 01028, US

(21) Appl. No.: 12/227,078

(22) PCT Filed: Aug. 22, 2007
(86) PCT No.: PCT/US07/18570

§ 371 Date: Nov. 6, 2008

Related U.S. Application Data
(63) Continuation of application No. 11/890,387, filed on Aug. 6, 2007, now abandoned.
(60) Provisional application No. 60/839,805, filed on Aug. 24, 2006.

(51) Int. Cl. A61N 5/067

(52) U.S. Cl. 607/89

(57) ABSTRACT

A high power density light delivery device is presented that uses an optical fiber with a diffuser attached at a distal end for use in, for example, photodynamic therapy (PDT) and laser induced thermotherapy (LITT). Uniform scattering in the diffuser section is obtained by either inducing differences in refractive index profiles of the core or in the core-cladding interface with the use of scattering centers or nano-voids. Nano-voids are created in the core or core-cladding interface by focusing high power laser energy or picosecond/femtosecond laser pulses on the quartz fiber material to induce defects. A special method is used that writes defects into or near to the core/cladding boundary through the jacket, without the necessity to first remove the jacket and then recoat the fiber. The method uses a wavelength that is highly transmissive in the jacket and fiber but absorbed in the fiber at very short laser pulses with high peak power. These processes allow the use of high power laser energy and the emission of the resulting high power densities in quartz fibers. The disclosed optical fiber delivery system is suitable for high power applications that require optical fibers with high flexibility and strength and core diameters from about 50 to about 400 microns.

### DOMESTIC PRIORITY UNDER 35 USC 119(e)

This application claims the benefit of U.S. Provisional Application Ser. No. 60/839,805 filed Aug. 24, 2006 and U.S. Full Application filed on Aug. 6, 2007, both of which are incorporated by reference herein.

### BACKGROUND OF THE INVENTION

1. Field of the Invention

The present invention relates to optical fiber diffusers, in general, and, in particular, to high power diff-users and methods of manufacturing such diff-users with scattering centers in the core or core-cladding interface.

2. Invention Disclosure Statement

Various medical and technical applications use optical fiber devices to deliver high energy light to specific locations and require a diffused light output at the intended treatment site. Consequently, most optical fiber devices have a dedicated means for controlling the output of radiant energy at the distal end of the optical fiber.

An optical fiber generally consists of an optically transmissive core and a cladding.

The cladding is typically surrounded by a protective jacket. Optical fibers have a cladding with a lower refractive index than the core to allow light to propagate to the end of the optical fiber. Light propagates along the fiber core as a result of total internal reflection at the interface between the fiber core and the cladding. However, achieving radial diffusion of the light at the distal end of the optical fiber requires either the addition of a diff-user or a changing of the fiber's physical characteristics.

Optical fiber diffusers have been successfully used in many industrial and medical applications for many years. Diffusers are generally used on the distal end of optical fibers as a means of directing and scattering the optical energy output therefrom as well as homogenizing the output. Uniform light emission using diffusers is well known in the art especially in the field of photodynamic therapy (PDT). As disclosed in the prior art, light diffusing optical fibers are conventionally made of plastic or glass and are limited to a low level power density.

For many potential applications of an optical fiber for illumination/irradiation purposes, the limitation of the output angle is a great disadvantage. In order to illuminate/irradiate a desired area being larger than an area that would. be covered by a conventional output from a fiber end, a diffuser is used. A diffuser is particularly useful in such applications in which it is desirable to heat, to illuminate or to irradiate an object uniformly in order to obtain uniform, predictable and reproducible results over some extended volume. There are several general methods discussed in prior art for producing a diffuser for optical fibers.

One of the simplest methods of constructing an axial diffuser from an, optical fiber cable is by removing the cladding layer(s) and then coating the resultant bare core with a layer of optical scattering material or scattering centers may be positioned on the core or cladding by roughening their surface. Roughening can be achieved in different ways, for example, either by chemical action, or by mechanical means such as scratching, abrading, or sanding the core or cladding layer. This method has several disadvantages in that diffusers which rely on a deformed core-to-cladding interface have the drawback of potentially weakening the mechanical properties of the fiber. In a medical application, an optical fiber diffuser used for internal irradiation involves bends, in which case the weakened area on the core caused by the roughening action can led to cracking or even breaking off of the diffuser. This can be overcome to some extent by having a protective layer around the exposed core such as in U.S. Pat. No. 6,361,530 by Mersch. But when a high energy in the range of several kilowatts is transmitted by the optical fiber, the protective layer at the outer periphery of cladding is often damaged by a temperature rise caused by energy leakage to the protective layer or by direct incidence of light on the protective layer.

Generally, in the prior art, a large diameter fiber is used for transmission of high power densities. Prior optical fiber diffusers used for high power transmission have core lo diameters in the range of about 800 to about 1500 μm and can carry light energies up to 4 Watts. In other words a power density in optical fiber diffuser with a core diameter of 1500 μm and power of 1 watt is 0.055 kW/cm² and for a power of 5 watts is 0.28 kW/cm². On the other hand, a power density in a diffuser with a core diameter of 800 μm and 1 watt power is 0.200 kW/cm²and for 5 watts power it is 1.00 kW/cm²; power density being inversely proportional to the square of the diameter of the core fiber. Due to increased power density in the fiber tips, heat damage is common among high power diffusers. Many high power diffusers have coolants attached to protect the fiber from heat damage.

U.S. Pat. No. 6,167,177 by Sandström et al. discloses an optical fiber with a quartz core and a glass or polymer cladding capable of transmitting high power exceeding 1 kW. The central core is surrounded by a cladding layer and the fiber end has cooling device attached about the fiber end. Special liquid coolant is used for cooling the optical fiber end and protecting the optical fiber end from heat damage. The fiber end is abutted to a window in the cooling device. Providing a coolant to the fiber end can increase the complexity of the optical fiber diffuser and certainly reduces its flexibility. Moreover, the potential for breakage of the coolant lines and the possibility of contaminating a patient is a significant drawback to this device.

U.S. Pat. No. 4,466,697 by Daniel discloses a method of making an optical fiber output which can transmit light uniformly therefrom. In the plastic core reflective light scattering particles are embedded in the core so that reflected light exits in a radial manner. It is further noted that bubbles may be formed in the plastic core by the use of laser radiation, but the use of glass fibers having such is not noted. The formation of bubbles in low melting point materials like plastic by applying radiant energy is well known in material science.

U.S. Pat. No. 5,536,265 by Van den Bergh et al. discloses a method of manufacturing light diffusers for radial emission of light. The diffuser tip is created by the removal of the cladding and roughening action thereby comprising mechanical integrity. The roughened tip is surrounded by elastic material and enclosed in an outer tube. A weakened area on the core is caused by the roughening action which can lead to cracking or even breaking off of the diffuser.

Some optical fibers diffuse light using microbeads or other Rayleigh scatterers which are distributed along the fiber tip. See, for example, U.S. Pat. No. 5,196,005 and U.S. Pat. No. 5,330,465. In these references, Doiron et al. describe a diffuser tip comprised of a silicone extension piece that has scattering centers embedded within it. The scattering centers are not uniformly distributed over the extension piece but rather increase in density towards the distal end of the diffuser tip. The disclosed silicone extension is a separate piece which is attached to the distal end of the fiber. Silicone may further be damaged by laser heat, impact or possible chemicals. Another device described is in U.S. Pat. No. 5,9789,541, which discloses a method for the radial distribution of light which requires depositing multiple layers of light scattering particles onto the core of the fiber at the distal tip after first stripping away the cladding. The resulting diffusion properties are customized by controlling the density of the scattering elements on the surface of the fiber. The stripping of the cladding layers also creates other problems like environmental issues.

Another example of a diffusing optical fiber is described by Gu et al. in WO 00/79319. They disclose an optical fiber diffuser comprised of a Bragg grating that is “written” onto the surface of the fiber using a UV laser. The disclosed Bragg grating is created using a phase mask to ensure that a highly regular interference pattern will be written onto the fiber surface in a point-to-point fashion. A similar technique of using tightly focused laser light to “mark” or engrave objects was recently described in Industrial Laser Solutions for Manufacturing, May 2004. This requires additional dopants in the core.

Other patents disclose processes of creating optical inhomogeneities in plastic and soft glass fibers by the use of lasers and heating but these are not appropriate for creating such scattering centers in quartz fibers that are typically formed by melting of a perform in a temperature range close to 2000° C. compared to 200 to 600° C. for these aforementioned materials. These fibers could not be used for high power laser energy densities contemplated by the present invention because of optical glass and the concomitant heating damage.

The diffusers discussed in prior art are limited in application because the underlying optical fiber is weakened by mechanical processing during its manufacture. Weakened optical fibers have limited flexibility and the output intensity of light energy is reduced which can lead to uncertain dosimetry and inconsistent results. Other drawbacks include non-uniform diffusion and complex manufacturing steps. The present state of art fails to disclose a diffuser which can transmit uniform radiation for high power densities through a small core diameter and also having additional characteristics like flexibility and uniform mechanical property throughout the fiber assembly.

It is thus desirable therefore to provide a high power optical fiber diffuser, which overcomes limitations in the prior art, and which contains a smaller core diameter, flexible optical fiber diffuser and reliable mechanical properties for the transmission and uniform distribution of high power light.

### OBJECTS AND SUMMARY OF THE INVENTION

It is therefore one objective of the present invention to provide an improved optical fiber diffuser for use in medical procedures involving the use of photodynamic therapy.

It is another objective of the present invention to provide an improved optical fiber diffuser for use in medical procedures that require high power densities.

A still further objective of the present invention is to provide an improved optical fiber diffuser for use in medical procedures that require high power densities and further use quartz fibers.

Another objective of the present invention is to provide diffusers having optical fibers with small core diameters from about 50 to about 200 micron, for example, and have the capacity to handle high power radiation of about 1 W/cm² to about 5 W/cm² while maintaining the strength and the mechanical properties of the fiber.

Briefly stated, a high power density light delivery device is presented that uses an optical fiber with a diffuser attached at a distal end for use in, for example, photodynamic therapy (PDT) and laser induced thermotherapy (LITT). Uniform or designed profile scattering in the diffuser section is obtained by either inducing differences in refractive index profiles of the core or in the core-cladding interface with the use of scattering centers or nano-voids. Nano-voids are created in the core or core-cladding interface by focusing high power laser energy or picosecond/femtosecond laser pulses on the quartz fiber material to induce defects. A special method is used that writes defects into or near to the core/cladding boundary through the jacket, without the necessity to first remove the jacket and then recoat the fiber. The method uses a wavelength that is highly transmissive in the jacket and fiber but absorbed in the fiber at very short laser pulses with high peak power. These processes allow the use of high power laser energy and the emission of the resulting high power densities in quartz fibers. The disclosed optical fiber delivery system is suitable for high power applications that require optical fibers with high flexibility and strength and core diameters from about 50 to about 400 microns.

The above and other objects, features and advantages of the present invention will become apparent from the following description read in conjunction with the accompanying drawings.

### BRIEF DESCRIPTION OF THE DRAWINGS

FIG. 1 illustrates by a cross sectional longitudinal view an optical fiber diffuser of one of the embodiments of the present Invention wherein the scattering centers are located in the core of the optical fiber.

FIG. 2 illustrates the embodiment of FIG. 1 wherein the diffuser is scattering light energy within the core of optical fiber diffuser of the present invention.

FIG. 3 illustrates by a cross sectional longitudinal view of the optical fiber delivery system using another embodiment of the diff-user that shows the scattering centers located in the core-cladding interface of the optical fiber, on the cylindrical surface within the optical fiber.

FIG. 4 illustrates by a cross sectional longitudinal view the diffuser of the present invention manufactured by a sol-gel technology with scattering particles in the cladding layer further including an end cap.

### DETAILED DESCRIPTION OF PREFERRED EMBODIMENTS

This invention relates to an improved optical fiber diffuser for use in the photodynamic therapy treatment of tumors and other medical conditions. Medical applications also need broad irradiation patterns and can benefit from small diameter fibers s to provide minimal invasive surgery. Presently, optical fiber diffusers used in the medical applications have core sizes in the range of 800 to 1500 μm; large diameter fibers are often used because of the power density capacity. When a power of 1 watt is transmitted through a fiber core of 1500 μm, the power density is 0.055 kW/cm²; and for 5 watts of power, the power density is 0.28 kW/cm². On the other hand a power density in a diffuser with a core diameter of 800 μm and 1 watt of power is 0.200 kW/cm² and for 5 watts of power it is 1.00 kW/cm².

In this invention, an optical fiber diffuser with core diameters from about 50 to about 400 micron is used to transmit power of 1 watt of CW laser energy or more. The power density experienced in a core diameter of 50 microns is 50 kW/cm² as compared to a core diameter of 400 microns being 0.80 kW/cm². (At the same time power density for 5 watts of power in 50 and 400 micron core diameter is 250 kW/cm² and 4.0 kW/cm² respectively). Fibers with good mechanical and physical properties are used for manufacturing high power diffusers because medical applications as well as most other applications using high power transmission requires excellent mechanical and physical stability.

In one of the preferred embodiments of the diffuser, the scattering centers are located in the core of the fiber in a predetermined length near the distal end. The scattering centers are intentionally created therein and are, for example, nanovoids which are created by focusing high power laser radiation thereon. The nanovoids can also be created at the core-cladding interface of a silica/silica structure. The nanovoids may be bubbles in the optical fiber or may be other defects having a change in refractive index as compared to the surrounding area. The size of the nanovoids is chosen in accordance with the wavelength that is to be scattered by the diffuser from the optical fiber.

In a preferred embodiment, the nanovoids in the core or core/cladding structure are created using picosecond or femtosecond laser pulses. The short laser pulse is focused through the circumference of the core. The laser energy is applied with sufficient power for a predetermined duration of time to create the defects required at sufficient distance from each other to provide a uniformly scattering diffuser.

The optical fibers in this invention must be capable of transmitting high power laser energy, and should have high flexibility and mechanical strength even at small core diameters of about 50 to 400 microns. High power applications are approximately 1 W (5 W) (CW or average power) per cm diffuser length and corresponds to a power density of 50 kW/cm² to 0.80 kW cm². Additionally, it is advantageous to apply the short laser pulses through the protective jacket of the fiber, particularly if the scattering centers are generated offline from the drawing tower.

In another example of this invention the diffuser with scattering particles is manufactured as a separate unit using a sol-gel process. This diffuser unit is then spliced onto a conventional fiber.

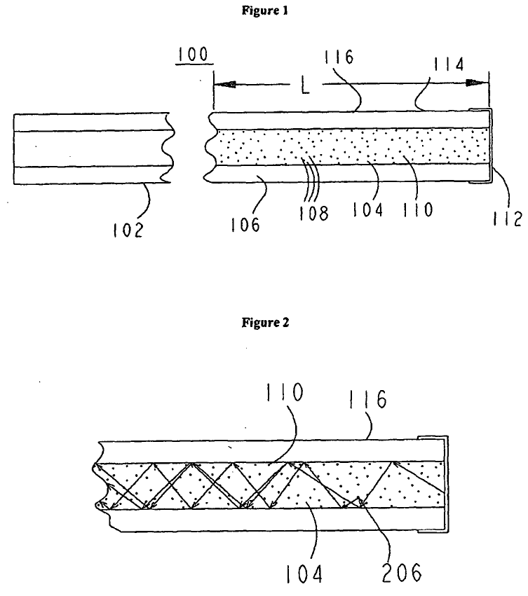

FIG. 1 illustrates by a partial longitudinal view optical fiber high power delivery system 100 capable of handling high powers of 1 watt and more. Optical fiber 102 comprises higher refractive index core 104 and lower refractive index cladding 106. Only one layer of cladding is shown in the present invention although additional layers of cladding may be present Diffuser 116 of the present invention consists of a predetermined length of core 104 with a plurality of scattering centers 108 as shown consisting of nanovoids 110 which initiate the scattering process in core 104 of optical fiber 102. In this embodiment, core 104 has scattering centers 108 along a predetermined length “L” at the distal end of optical fiber 102. Scattering centers 108 in core 104 are created by focusing high power laser radiation during the drawing process of the optical fiber to create naonvoids 110. Terminal cap 112 may be added to distal end 114 to enhance the scattering process by reflecting back radiant energy reaching distal end 114.

Optical fiber diffuser 116 illustrated in FIG. 1 has a core diameter of about 50 to about 400 μm and may transmit high powers of 1 W (5 W) CW and above. Core 104 and cladding 106 are composed of, preferably, quartz or other silica materials which are capable of handling high power densities. The power density in this optical fiber 102 is 50 kw/cm² to 0.80 kw/cm², respectively, for the diameters noted. Power density in a 50 micron diameter core is sixty four times the power density in a 400 micron core or about 400× larger in a 1000 μm fiber.

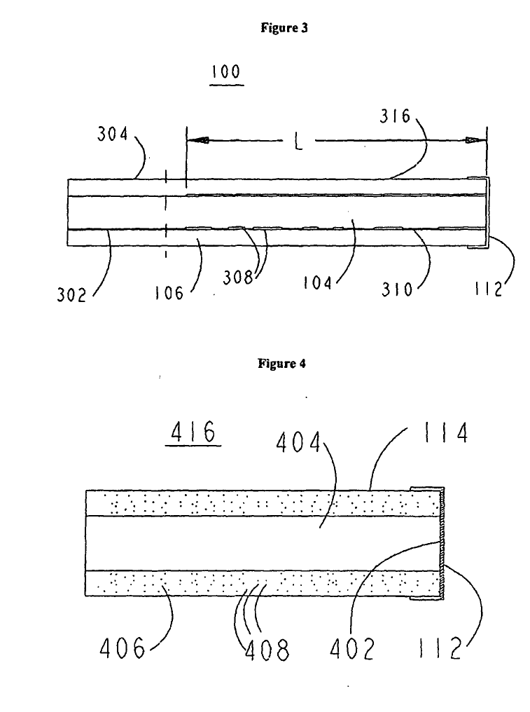

In another embodiment of this invention, FIG. 3, optical fiber diffuser 316 of optical fiber high power delivery system 100 has scattering centers 308 consisting of nanovoids 310 located in core-cladding interface 302 of optical fiber 304. Core 104 and cladding 106 are composed of quartz or of a silica material conventional in the art. Scattering centers 308 in core cladding interface 302 is of a predetermined length of ‘L’ as shown in the FIG. 3. Cap 112 may be further added to the distal end of the optical fiber.

FIG. 2 shows the scattering process in core 104 of diffuser 116 of FIG. 1. Scattering centers with nanovoids 110 inside core 104 are shown scattering light rays propagating through core 104 by total internal reflection. Light ray 206 depicts a ray which is scattered by a nanovoid.

In another preferred embodiment of diffuser 416 as seen in FIG. 4, diffuser 416 is manufactured separately by a sol-gel technology. FIG. 4 shows optical fiber diffuser 416 with central core 404 with surrounding cladding layer 406 of low refractive index material. Mirror 402 may be located at distal end 114 of diffuser 416 to reflect back any ray which reaches distal end 114 so that uniform diffusion of light is made possible. Further end cap 112 may be added thereon also. In the sol-gel process, scattering particles of TiO₂ 408 are incorporated into cladding layer 406 which enhances the scattering process. The size and distribution of the scattering particles may be adjusted appropriately during manufacture depending on the wavelength of light to be scattered. Separate diffuser 416 is attached to an end surface of the optical fiber of the delivery system in a conventional manner to minimize reflections or other losses of radiation.

Although scattering centers have been noted as voids such as bubbles or scattering particles, other scattering centers may result from modifying the refractive indexes in a volume as compared to the surrounding material.

By selecting a proper density of the nano voids, the overall length of the active scattering portion of the diffuser can be designed by having a certain profile that does not have to be homogeneous, but can be adapted to the intended application. In the manufacturing process by writing with short pulse lasers, the laser pulse energy and pulse length determine the effective size of the void; and the number of pulses determines the density of the voids in the irradiated area.

While the benefits of the present invention are the ability to achieve and deliver high power densities through use of small diameter optical fibers, use of these small fibers and special diff-users can also benefit applications, such as photodynamic therapy, where modest power densities and designed stereographic emission profiles are desirable.

Having described preferred embodiments of the invention with reference to the accompanying drawings, it is to be understood that the invention is not limited to the precise embodiments, and that various changes and modifications may be effected therein by a person skilled in the art without departing from the scope or spirit of the invention according to the appended claims.

What is claimed is:

1. An optical fiber delivery system for use in medical procedures requiring high power densities comprising
at least one optical fiber having a core and at least one lower refractive index cladding about said core, said at least one optical fiber having a proximal and distal end, a source of optical energy inputting said optical energy into said proximal end of said at least one optical fiber;
a diffuser for outputting high power density optical energy to a treatment site from said distal end of said at least one optical fiber, said diffuser being a section of a predetermined length of said distal end of said at least one optical fiber; and
scattering centers being positioned in said section of said predetermined length at said distal end of said optical fiber, said scattering centers causing a portion of said inputted optical energy to exit radially on to a treatment site.

2. The optical fiber delivery system according to claim 1 wherein said scattering centers are located in said predetermined length of said fiber core or in or near an interface between said fiber core and said cladding in said predetermined length.

3. The optical fiber delivery system according to claim 2 wherein said scattering centers are defects such as nanocracks or nano-voids creating localized refractive index differentials in either said core or in or near said interface between said core and said cladding.

4. The optical fiber delivery system according to claim 2 wherein said scattering centers are scattering particles included within said core or said cladding of said core.

5. The optical fiber delivery system according to claim 4 wherein said scattering particles are positioned in said optical fiber by a sol-gel technique.

6. The optical fiber delivery system according to claim 1 wherein said scattering centers are distributed in size and frequency and location to accomplish a uniform intensity distribution over said predetermined length with or without a partial or totally reflecting element or mirror at said fiber's distal end.

7. The optical fiber delivery system according to claim 1 wherein the mechanical properties are essentially equal in said optical fiber and in said diffuser.

8. The optical fiber delivery system according to claim 1 wherein said diffuser containing said scattering centers is attached to a distal end of said optical fiber utilizing high strength splicing procedures to said distal end of a conventional fiber to form said diffuser system.

9. The optical fiber delivery system according to claim 1 further including a cap over said distal end of said diffuser to facilitate and/or enhance scattering output.

10. The optical fiber delivery system according to claim 1 suitable to handle high power levels and a scattering output end of a defined length characterized by a solid core/clad structure of quartz glass or doped quartz glass and scattering being initiated in said core or at said core-cladding interface.

11. A method of performing a medical procedure at a treatment site using an optical fiber delivery system according to claim 1 and a radiation source.

12. The method of performing a medical procedure according to claim 11 wherein said medical procedure includes photodynamic therapy or interstitial thermo therapy using an optical fiber delivery system as claimed in claim 1 with a photosensitizer and a radiation source with a suitable wavelength or spectrum.
